(19) United States
(12) Patent Application Publication
(10) Pub. No.: US 2003/0147957 A1
(43) Pub. Date: Aug. 7, 2003

(54) Dual release formulation comprising levodopa ethyl ester and a decarboxylase inhibitor in immediate release layer with levodopa ethyl ester and a decarboxylase inhibitor in a controlled release core

(76) Inventors: Daniela Licht, Ramat Ilan Givat Shmuel (IL); Shulamit Patashnik, Reut (IL); Ezmira Naftali, Rosh-Ha'ayin (IL); Naim Sayag, Yuvalim (IL); Adrian Gilbert, Ra'anana (IL); Sasson Cohen, Tel Aviv (IL); Corinne Zollmann, Kochav Yair (IL)

Correspondence Address: Cooper & Dunham LLP, 1185 Avenue of the Americas, New York, NY, 10036, US

(21) Appl. No.: 10/195,036

(22) Filed: Jul. 12, 2002

Related U.S. Application Data
(60) Provisional application No. 60/346,744, filed on Jan. 7, 2002.
(60) Provisional application No. 60/305,179, filed on Jul. 12, 2001.

(51) Int. Cl. A61K 9/24

(52) U.S. Cl. 424/471

(57) ABSTRACT

A tablet which comprises:

an inner core formulated for controlled release comprising a mixture of

(a) a granulated admixture of a decarboxylase inhibitor and a surfactant, and

(b) levodopa ethyl ester or a derivative or a pharmaceutically acceptable salt thereof; and

an outer layer encapsulating the inner core and formulated for immediate release comprising a mixture of a granulated decarboxylase inhibitor and levodopa ethyl ester or a derivative or a pharmaceutically acceptable salt thereof. The subject invention also encompasses a method of treating patients suffering from Parkinson's disease or related disorders by the administration of the pharmaceutical compositions of the subject invention. Additionally, the subject invention provides methods of manufacturing the tablets of the subject invention.

This application claims the benefit of U.S. Provisional Application No. 60/346,744, filed Jan. 7, 2002, and U.S. Provisional Application No. 60/305,179, filed Jul. 12, 2001, both of which are hereby incorporated by reference.

Throughout this application, various references are referenced by citations within parenthesis. These references, in their entireties, are hereby incorporated by reference to more fully describe the state of the art to which this invention pertains.

### FIELD OF THE INVENTION

This invention relates to the treatment of Parkinson's disease (PD) and related disorders with levodopa (L-DOPA) and a decarboxylase inhibitor.

### BACKGROUND OF THE INVENTION

Parkinsonian patients are routinely treated with a combination of levodopa (L-DOPA) and a decarboxylase inhibitor such as carbidopa or benserazide (see, e.g. U.S. Pat. Nos. 6,218,566, 5,525,631 and 5,354,885). Unfortunately, after an initial period of satisfactory, smooth and stable clinical benefits from L-DOPA therapy lasting on the average 2-5 years, the condition of many patients deteriorate and they develop complex dose-related as well as unpredictable response fluctuations. The causes of the response fluctuations are probably multiple and complex, but pharmacokinetic problems (primarily faulty absorption of L-DOPA) may play a critical role. There is a correlation between the clinical fluctuations and the oscillations of L-DOPA plasma levels. Many of the problems are a result of the unfavorable pharmacokinetic properties of L-DOPA, i.e., very poor solubility, poor bioavailability and short half-life in vivo.

A typical problem for Parkinsonian patients is the “on-off” oscillations in which daily motor activity is dominated by remarkable swings between “off” hours, when they are severely incapacitated, rigid, unable to move and sometimes to speak or swallow, to “on” periods where they are responsive to L-DOPA and can, more or less, perform. The current treatments (apomorphine, lisuride) used to treat patients in the “off” period are unsatisfactory.

Various procedures have been attempted to remedy this situation. In some cases, direct instillation of a slurry of levodopa through a duodenal tube has given rapid relief from the “off” state (Durlan R. et al. (1986), Ann. Neurol.20: 262-265 and Cedarbaum et al. (1990), Neurology 40:878-995). In another approach, oral dosing with a dilute aqueous solution of levodopa appeared to be effective (Kurth M. C. et al. (1993), Neurology 43:1036-1039). However, neither of these measures are practical enough to allow self-medication when urgently needed. When rapid relief is needed, the more common procedure is to recommend the patients to crush the levodopa tablet before intake so as to minimize the time required for its disintegration in the gastrointestinal (GI) tract. The efficacy of this procedure has never been demonstrated.

A major problem in long-term treatment of PD with chronic intermittent levodopa therapy is fluctuating motor response—the “on-off” phenomenon and the increasingly frequent appearance of dyskinesia. There is some evidence that these often quite disturbing variations in drug response are due, in part, to fluctuations in drug plasma concentration which is responsible for the early (but temporary) severe dyskinetic bouts and the quickly dropping plasma levels may well be the cause of premature “end of dose” aggravations of the motor disability.

An important approach to the treatment of those phenomena is the attempt to prolong the duration of levodopa in the plasma with the use of sustained, controlled-release (CR) preparations. Such a dosage form is currently available under the brand name SINEMET CR® (Merck Sharpe & Dohme), which is a compressed tablet containing controlled release carbidopa-levodopa. This formulation produces a constant rise in plasma levodopa level that is sustained for 3 to 4 hours, which is significantly longer than that obtained with immediate-release carbidopa-levodopa preparations. An initial absorption phase is lacking, but a gradual build-up in the absorption profile occurs. Peak levels are not recorded until after 2 hours. SINEMET CR® is available in two forms: 1) SINEMET CR 50-200®, containing 50 mg carbidopa and 200 mg levodopa; and 2) SINEMET CR 25-100, containing 25 mg carbidopa and 100 mg levodopa.

Controlled-release carbidopa-levodopa, such as SINEMET CR®, has approximately 30% less bioavailability compared to immediate-release preparations, such as SINEMET®. Considerable inter-subject variation has been observed in levodopa absorption. Peak levodopa plasma levels following administration of controlled-release carbidopa-levodopa are lower than those found with immediate-release preparations.

A number of open-label studies of controlled-release carbidopa-levodopa in PD patients with motor fluctuations have demonstrated a significant reduction in “off” time and improvement in clinical disability scores. Goetz et al. compared the controlled-release formulation of carbidopa-levodopa to the immediate-release preparations in an open-label trial in 20 PD patients with “wearing-off” phenomena and found increased “on” time after 4 to 6 weeks of therapy ((1987), Neurology 37: 875-878). In 9 patients followed for 3 months, the “on” time” without chorea remained significantly increased. In another open label study of 17 PD patients with severe fluctuations, controlled-release carbidopa-levodopa, when compared with the immediate-release formulation, caused a reduction in the number of “off” periods and a slight increase in “on” time. Some patients required additional immediate-release carbidopa-levodopa.

Friedman and Lannon noted that “wearing-off” but not “on-off” phenomena improved in 19 patients in a 1-year open-label study ((1989), Clinical Neuropharmacol.12: 220-223). In 20 PD patients with motor fluctuations treated with controlled-release carbidopa-levodopa for one year, Rondot et al. reported an improvement in clinical scores and a 63% prolongation of “on” periods ((1989), Neurology. 39(suppl 2): 74-77).

In a 52-week open-label trial of 20 patients, it was noted that with controlled-release carbidopa-levodopa, fluctuations became less troublesome, but did not disappear (Aarli, J. A., Gilhus, N. E. (1989), Neurology. 39 (suppl 2): 82-85). In an open-label trial involving eight PD patients with fluctuations given controlled-release carbidopa-levodopa for 36 to 39 months, five patients experienced an increase in daily “on” time when compared with baseline (Rodnitzki, R. L. et al. (1989), Neurology. 39 (suppl 2): 92-95)

Pahwa et al. converted 158 patients from immediate-release to controlled-release carbidopa-levodopa ((1993), Neurology. 43: 677-681) and found that the “off” time decreased significantly. 73% of the patients preferred the controlled-release preparation; Pahwa et al. concluded that controlled-release carbidopa-levodopa was particularly effective in decreasing motor fluctuations in PD patients with mild-to-moderate disease. In a study of 17 patients with motor fluctuations, immediate-release and controlled-release carbidopa-levodopa were compared over several doses during one day. During treatment with the controlled-release preparation, total “on” time was increased, and the number of “off” episodes was reduced.

It appears that controlled-release carbidopa-levodopa preparations provide a more stable and constant levodopa plasma level than immediate-release formulations. Controlled-release preparations are efficacious in the treatment of motor fluctuations in PD, have longer duration of action for each dose, cause a decrease in dose failures, a reduction in early morning dystonia and a decrease in nocturnal awakenings.

However, controlled-release preparations also cause a slower or delayed onset of effect in some PD patients, which is related to the slow build-up of plasma levels of levodopa in the first dose. Therefore, some patients require an immediate-release preparation before taking the controlled-release preparation, especially for the first morning dose.

Rubin (U.S. Pat. No. 6,238,699 B1) discloses a pharmaceutical composition containing carbidopa and levodopa in immediate and controlled release compartments. Rubin teaches that his compositions may fall into any one of the following types: 1) a compressed inner tablet core onto which an outer tablet core is compressed (dual compression); 2) a capsule or compressed tablet containing pellets; or 3) a layer tablet comprising two or more layers (sandwich). However, Rubin does not describe how to obtain an effective formulation with any agent other than levodopa. Thus, Rubin does not teach how to formulate a tablet having two drugs with very different solubilities (Table 1).

As an alternative to levodopa, Chiesi et al. disclose pharmaceutical compositions comprising controlled release and immediate release formulations of levodopa methyl ester and carbidopa (WO 99/17745). Chiesi et al. suggest the prepartation of their pharmaceutical compositions as 3-layer monolithic tablets (sandwiches). Chiesi et al. provide no guidance concerning how to formulate compositions other than composition containing levodopa methyl ester. In one formulation, the slow release layer contains levodopa methyl ester, but not carbidopa, while the remaining layers employ both levodopa methyl ester and carbidopa. However, a significant detriment to the utility of the compositions of Chiesi et al. is that levodopa methyl ester is metabolized into L-DOPA and methanol, of which methanol is toxic (U.S. Pat. No. 5,354,885).

Another replacement for L-DOPA is levodopa ethyl ester (LDEE). LDEE increases the bioavailability of L-DOPA due to its greater solubility (U.S. Pat. No. 5,354,885, Milman et al.). LDEE was incorporated into pharmaceutical compositions by Cohen et al. (U.S. Pat. No. 5,840,756). Although Cohen et al. state that their pharmaceutical compositions provide “a burst of levodopa followed by the maintenance of a sustained level of levodopa” (from the metabolism of levodopa ethyl ester), the compositions are only controlled release compositions (see Example 2). They disclose that their compositions may be formulated as single compression tablets and may contain a decarboxylase inhibitor. Cohen et al., however, offer no guidance impetus for formulating a composition where the decarboxylase inhibitor and LDEE are released at an approximately equal rate.

Levin (WO 00/27385) also combined levodopa ethyl ester and a decarboxylase inhibitor, carbidopa, in pharmaceutical compositions. However, in contrast to Cohen et al., the pharmaceutical compositions disclosed by Levin are solely immediate release formulations.

Thus, there is a need for an LDEE pharmaceutical composition with a decarboxylase inhibitor that will increase the bioavailability of levodopa. Such a pharmaceutical composition needs to dissolve rapidly in a patient requiring levodopa therapy, and at the same time, provide a sustained therapeutic level of levodopa in the patient, have good patient compliance and be easy to manufacture.

### SUMMARY OF THE INVENTION

The subject invention provides a tablet which comprises:

an inner core formulated for controlled release comprising a mixture of

(a) a granulated admixture of a decarboxylase inhibitor and a surfactant, and

(b) levodopa ethyl ester or a derivative or a pharmaceutically acceptable salt thereof; and

an outer layer encapsulating the inner core and formulated for immediate release comprising a mixture of a granulated decarboxylase inhibitor and levodopa ethyl ester or a derivative or a pharmaceutically acceptable salt thereof.

The subject invention further provides a tablet which comprises:

an inner core formulated for controlled release comprising a mixture of

(a) a granulated admixture of from above 0 mg up to about 100 mg carbidopa, and from about 0.03 mg to about 75 mg of a surfactant,

(b) from about 4 mg up to about 400 mg levodopa ethyl ester or a derivative or a pharmaceutically acceptable salt thereof, and

(c) an inner core excipient component; and

an outer layer encapsulating the inner core and formulated for immediate release comprising a mixture of:

(i) from above 0 mg up to about 75 mg carbidopa,

(ii) from about 5 mg up to about 300 mg levodopa ethyl ester or a derivative or a pharmaceutically acceptable salt thereof, and

(iii) an outer layer excipient component.

In addition, the subject invention provides a tablet which comprises an inner core formulated for controlled release comprising a mixture of

(a) a granulated admixture of above 0 mg up to about 100 mg carbidopa, from about 0.03 mg to about 75 mg of a surfactant and at least one excipient,

(b) from about 4 mg up to about 400 mg levodopa ethyl ester or a derivative or a pharmaceutically acceptable salt thereof, and

(c) an inner core excipient component;

and an outer layer encapsulating the inner core and formulated for immediate release comprising a mixture of

(i) a granulated admixture of from about 1 mg up to about 75 mg carbidopa and at least one excipient; and

(ii) from about 5 mg up to about 300 mg levodopa ethyl ester or a derivative or a pharmaceutically acceptable salt thereof, and

(iii) an outer layer excipient component.

The subject invention also provides a method of treating a subject suffering from a disease selected from the group consisting of Parkinson's disease, senile dementia, dementia of the Alzheimer's type, a memory disorder, depression, hyperactive syndrome, an affective illness, a neurodegenerative disease, a neurotoxic injury, brain ischemia, a head trauma injury, a spinal trauma injury, schizophrenia, an attention deficit disorder, multiple sclerosis, withdrawal symptoms, epilepsy, convulsions and seizures, which comprises administering to the subject the tablet of the subject invention in an amount effective to treat the disease.

Furthermore, the subject invention provides methods of manufacturing the tablets of the subject invention.

### DETAILED DESCRIPTION OF THE INVENTION

The subject invention provides a tablet which comprises:

an inner core formulated for controlled release comprising a mixture of

(a) a granulated admixture of a decarboxylase inhibitor and a surfactant, and

(b) levodopa ethyl ester or a derivative or a pharmaceutically acceptable salt thereof; and

an outer layer encapsulating the inner core and formulated for immediate release comprising a mixture of a granulated decarboxylase inhibitor and levodopa ethyl ester or a derivative or a pharmaceutically acceptable salt thereof.

As used herein, a “derivative of levodopa ethyl ester” is a compound that has substantially the same effect as levodopa ethyl ester in the treatment of Parkinson's disease and related disorders. Derivatives of levodopa ethyl ester includes compounds having structures such as those disclosed in U.S. Pat. No. 4,873,263.

A pharmaceutically acceptable salt of levodopa ethyl ester is any pharmaceutically acceptable salt of levodopa ethyl ester, e.g., the hydrochloride salt, the octanoate salt, the myristate salt, the succinate salt, the succinate dihydrate salt, the fumarate salt, the fumarate dihydrate salt, the acetate salt, the mesylate salt, the esylate salt, the tartarate salt, the hydrogen tartarate salt, the benzoate salt, the phenylbutyrate salt, the phosphate salt, the citrate salt, the ascorbate salt, the mandelate salt, or the adipate salt of levodopa ethyl ester.

As used herein, the phrase, “controlled release” means the release of small increments over time, usually requiring several hours to achieve 100% dissolution. Controlled release formulations encompasses, for example, slow release, extended release and sustained release formulations.

As used herein, the phrase, “immediate release” indicates that the drug is allowed to dissolve in the gastrointestinal contents, with no intention of delaying or prolonging the dissolution or absorption of the drug (FDA Guidance for Industry SUPAC-MR: modified release oral dosage forms CDER, September, 1997). Immediate release formulations encompass, for example, rapid burst formulations.

In the tablet, the decarboxylase inhibitor may be carbidopa.

In one embodiment, the surfactant is an ionic surfactant. In another embodiment, the surfactant is an aionic surfactant.

Additionally, the subject invention provides a tablet which comprises:

an inner core formulated for controlled release comprising a mixture of

(a) a granulated admixture of from above 0 mg up to about 100 mg carbidopa, and from about 0.03 mg to about 75 mg of a surfactant,

(b) from about 4 mg up to about 400 mg levodopa ethyl ester or a derivative or a pharmaceutically acceptable salt thereof, and

(c) an inner core excipient component; and

an outer layer encapsulating the inner core and formulated for immediate release comprising a mixture of:

(i) from above 0 mg up to about 75 mg carbidopa,

(ii) from about 5 mg up to about 300 mg levodopa ethyl ester or a derivative or a pharmaceutically acceptable salt thereof, and

(iii) an outer layer excipient component.

In one embodiment, in the outer layer, the carbidopa in (i) comprises granulated carbidopa.

In one embodiment, the surfactant is an ionic surfactant. In another embodiment, the surfactant is an aionic surfactant.

In another embodiment, the inner core is formulated such that the rate of release of the carbidopa is substantially the same as the rate of release of the levodopa ethyl ester or derivative or pharmaceutically acceptable salt thereof.

In this application, the phrase “substantially the same as” with reference to comparison of rates of release of components of the inner core or of components from the outer layer of a tablet means that the rates of release of the compounds being compared are the same, or if the rates differ, they differ by less than 35% between or among the components being compared.

In a further embodiment, the outer layer is formulated such that the rate of release of the carbidopa is substantially the same as the rate of release of the levodopa ethyl ester or derivative or pharmaceutically acceptable salt thereof.

In an added embodiment, the inner core is formulated such that the rate of release of the carbidopa is substantially the same as the rate of release of the levodopa ethyl ester or derivative or pharmaceutically acceptable salt thereof.

In yet another embodiment, the inner core excipient component comprises an excipient used as a carrier. Non-limiting examples of a carrier (extended release agent) used in the subject invention (used for example for the controlled release) are cellulose acetate, glyceryl monostearate, zein, microcrystalline wax, hydroxymethylcellulose, hydroxyethylcellulose, hydroxypropylmethylcellulose, carboxyvinyl polymers, polyvinyl alcohols, glucans, scleroglucans, chitosans, mannans, galactomannans, amylose, alginic acid and salts and derivatives thereof, acrylates, methacrylates, acrylic/methacrylic copolymers, polyanhydrides, polyaminoacids, methyl vinyl ethers/maleic anhydride copolymers, carboxymethylcellulose and derivatives thereof, ethylcellulose, methylcellulose and cellulose derivatives in general, modified starch and polyesters, polyethylene oxide.

In a preferred embodiment, the excipient used as a carrier comprises a hydroxypropylmethylcellulose. In another preferred embodiment, the hydroxypropylmethylcellulose has an average molecular weight between about 10 kDa and about 1500 kDa. In a further preferred embodiment, the hydroxypropylmethylcellulose has 19%-24% methoxyl substituent and 7%-12% hydroxylproproxyl substituent. In an added embodiment, the hydroxypropylmethylcellulose has a particle size distribution such that about 100% of the hydroxypropylmethylcellulose passes through a 30 mesh screen. In one embodiment, the hydroxypropylmethylcellulose has a particle size distribution such that about 99% of the hydroxypropylmethylcellulose passes through a 40 mesh screen. In yet another embodiment, the hydroxypropylmethylcellulose has a particle size distribution such that 55%-95% of the hydroxypropylmethylcellulose passes through a 100 mesh screen. In a further embodiment, the hydroxypropylmethylcellulose has a particle size distribution such that 65%-85% of the hydroxypropylmethylcellulose passes through a 100 mesh screen. In an additional embodiment, the hydroxypropylmethylcellulose has a particle size distribution such that about 80% of the hydroxypropylmethylcellulose passes through a 100 mesh screen. In a further embodiment, the hydroxypropylmethylcellulose has a particle size distribution such that about 90% of the hydroxypropylmethylcellulose passes through a 100 mesh screen. In a preferred embodiment, the hydroxypropylmethylcellulose is a Methocel®, such as Methocel KLOOLVP® (also known as Methocel KLOOLV®) or Methocel K15 MP® (also known as Methocel K15M®).

In one embodiment, the outer layer excipient component and/or the inner core excipient component comprises an excipient used as a binding agent. Non-limiting examples of a binding agent used in the subject invention (used for example for the granulate) are alginic acid, acia, carbomer, carboxymethylcellulose sodium, dextrin, ethylcellulose, gelatin, guar gum, hydrogenated vegetable oil, hydroxyethylcellulose, hydroxypropylcellulose (e.g., Klucel®), hydroxypropylmethylcellulose, liquid glucose, magnesium aluminum silicate, maldodextrin, methylcellulose, polymethacrylates, povidone, pregelatinized starch, sodium alginate, starch, and zein. In a preferred embodiment, the excipient used as a binding agent comprises a hydroxypropylcellulose.

In an additional embodiment, the outer layer excipient component comprises an excipient used as a disintegrating agent. Non-limiting examples of a disintegrant used in the subject invention (used for example for the disintegration of immediate release tablet) are kaolin, starch, powdered sugar, sodium starch glycolate, crosscarmelose sodium, carboxymethylcellulose, microcrystalline cellulose and sodium alginate. In a preferred embodiment, the excipient used as a disintegrating agent comprises a starch. In another preferred embodiment, the starch is a partially pregelatinized maize starch, such as Starch 1500®.

In yet another embodiment, the inner core excipient component and the outer layer excipient component each comprise an excipient useful as a flow agent and/or an excipient useful as a lubricant.

In one embodiment, the excipient useful as a flow agent comprises a micron-sized silica powder. A non-limiting example of a flow agent used in the subject invention (used for better flow of the mix for compression) is colloidal silicon dioxide or Syloid®, which is a preferred embodiment.

Non-limiting examples of a lubricant used in the subject invention (used for example for better compression properties) are talc, sodium stearyl fumarate, magnesium stearate, calcium stearate, hydrogenated castor oil, hydrogenated soybean oil and polyethylene glycol (PEG) or combinations thereof. In a preferred embodiment, the excipient useful as a lubricant comprises magnesium stearate. In another preferred embodiment, the excipient useful as a lubricant comprises sodium stearyl fumarate.

In a further embodiment, the inner core excipient component and the outer layer excipient component each comprise an excipient useful as a lubricant.

In another embodiment, the same excipient useful as a lubricant is present in both the inner core excipient component and the outer layer excipient component.

In an additional embodiment, the excipient useful as a lubricant present in the outer layer excipient component comprises sodium stearyl fumarate.

In a further embodiment, the excipient useful as a lubricant present in the inner core excipient component comprises sodium stearyl fumarate.

In yet another embodiment, the inner core excipient component comprises a first excipient useful as a lubricant and a second excipient useful as a lubricant. In a preferred embodiment, the first excipient usesful as a lubricant is sodium stearyl fumarate and the second excipient useful as a lubricant is magnesium stearate.

In one embodiment, the inner core excipient component and/or the outer layer excipient component comprises an excipient useful as a filler. Fillers may be inorganic or organic materials, and may be soluble or insoluble. Non-limiting examples of a filler used in the subject invention (used for example for weight adjustment and for better compression) are corn starch, lactose, glucose, various natural gums, methylcellulose, carboxymethylcellulose, microcrystalline cellulose, calcium phosphate, calcium carbonate, calcium sulfate kaolin, sodium chloride, powdered cellulose, sucrose, mannitol and starch. In a preferred embodiment, the excipient useful as a filler comprises a microcrystalline cellulose. In a more preferred embodiment, the microcrystalline cellulose has an average particle size between about 50 and about 90 microns. In a preferred embodiment, the microcrystalline cellulose is Avicel PH 101®, which has an average particle size of 50 microns. In another preferred embodiment, the microcrystalline cellulose is Avicel PH 112®, which has an average particle size of 90 microns.

In one embodiment, the inner core excipient component comprises an excipient used as a wetting agent, which may be an ionic or aionic (non-ionic) surfactant. Non-limiting examples of a wetting agent used in the subject invention (used for example for better dissolution of the active material) are sodium lauryl sulfate, benzalkonium chloride, benzethonium chloride, ducusate sodium, poloxamer, polyoxyethylene alkyl ethers, polyoxyethylene castor oil derivatives, polyoxyethylene sorbitan fatty acid esters, and polyoxyethylene stearates, cyclodextrin and derivatives. In a preferred embodiment, the amount of the wetting agent is not more than 10 mg. In another preferred embodiment, the wetting agent is an ionic surfactant. In still another preferred embodiment, the ionic surfactant is sodium lauryl sulfate.

In another embodiment, in the inner core

the granulated admixture in (a) comprises from above 0 mg up to about 75 mg carbidopa, and from about 0.03 mg up to about 50 mg of an ionic surfactant,

the levodopa ethyl ester or derivative or pharmaceutically acceptable salt thereof in (b) is present in an amount from about 10 mg up to about 400 mg;

and wherein in the outer layer,

the granulated carbidopa in (i) comprises from above 0 mg to about 75 mg carbidopa, and

the levodopa ethyl ester or derivative or pharmaceutically acceptable salt thereof in (ii) is present in an amount from about 10 mg up to about 250 mg.

In yet another embodiment, in the inner core

the ionic surfactant is present in an amount from about 0.1 mg up to about 50 mg, and

the levodopa ethyl ester or derivative or pharmaceutically acceptable salt thereof is present in an amount from about 50 mg up to about 400 mg;

and wherein in the outer layer

the granulated carbidopa comprises from about 10 mg up to about 50 mg carbidopa, and

the amount of the levodopa ethyl ester or the derivative or pharmaceutically acceptable salt thereof is from about 50 mg up to about 200 mg.

In a further embodiment, in the inner core

the granulated admixture comprises from 4.2 mg up to about 50 mg carbidopa and from about 0.1 mg up to about 10 mg of an ionic surfactant, and

the levodopa ethyl ester or derivative or pharmaceutically acceptable salt thereof is present in an amount from about 19 mg up to about 228 mg;

and wherein in the outer layer

the granulated carbidopa comprises from about 4.2 mg up to about 50 mg carbidopa, and

the levodopa ethyl ester or derivative or pharmaceutically acceptable salt thereof is present in an amount from about 19 mg up to about 228 mg.

In one embodiment, above 5% of the total levodopa ethyl ester or derivative or pharmaceutically acceptable salt thereof present in the tablet is in the outer layer.

In another embodiment, above 10% of the total levodopa ethyl ester or derivative or pharmaceutically acceptable salt thereof present in the tablet is in the outer layer.

In an additional embodiment, above 30% of the total levodopa ethyl ester or derivative or pharmaceutically acceptable salt thereof present in the tablet is in the outer layer.

In one embodiment, above 50% of the total levodopa ethyl ester or derivative or pharmaceutically acceptable salt thereof present in the tablet is in the outer layer.

In a further embodiment, above 70% of the total levodopa ethyl ester or derivative or pharmaceutically acceptable salt thereof present in the tablet is in the outer layer.

In yet another embodiment, above 10% of the total carbidopa present in the tablet is in the outer layer.

In one embodiment, above 30% of the total carbidopa present in the tablet is in the outer layer.

In an added embodiment, above 50% of the total carbidopa present in the tablet is in the outer layer.

In still another embodiment, above 70% of the total carbidopa present in the tablet is in the outer layer.

In a further embodiment, the tablet comprises about 342.0 mg total of levodopa ethyl ester or derivative or pharmaceutically acceptable salt thereof, and about 75.0 mg total of carbidopa.

In one embodiment, the tablet comprises about 228.0 mg total of levodopa ethyl ester or derivative or pharmaceutically acceptable salt thereof, and about 50.0 mg total of carbidopa.

In an added embodiment, the tablet comprises about 114.0 mg total of levodopa ethyl ester or derivative or pharmaceutically acceptable salt thereof, and about 25.0 mg total of carbidopa.

In yet another embodiment, the tablet comprises about 57.0 mg total of levodopa ethyl ester or derivative or pharmaceutically acceptable salt thereof, and about 12.5 mg total of carbidopa.

In an additional embodiment, in the inner core

the granulated admixture comprises sodium lauryl sulfate as the ionic surfactant and further comprises above 0 mg up to about 150 mg of a microcrystalline cellulose and from about 1 mg to about 50 mg of a hydroxypropylcellulose,

the 4 mg up to about 400 mg levodopa ethyl ester or derivative or pharmaceutically acceptable salt therof is present, and

the inner core excipient component comprises from about 2.5 mg up to about 245 mg hydroxypropylmethyl cellulose, above 0 up to about 150 mg of a microcrystalline cellulose, from about 1 mg to about 10 mg of a micron-sized silica and from about 1 mg to about 30 mg sodium stearyl fumarate and/or magnesium stearate,

and wherein in the outer layer

the granulated carbidopa is present in an amount from about 1 mg up to about 50 mg, and is present in a granulated admixture with 0 mg up to about 300 mg of a microcrystalline cellulose, from above 0 mg up to about 150 mg of a partially pregelatinized maize starch and from above 0 mg up to about 50 mg of a hydroxypropylcellulose,

about 114 mg levodopa ethyl ester is present, and

the outer layer excipient component comprises about 80 mg of a microcrystalline cellulose, about 4 mg of a partially pregelatinized maize starch, about 3 mg of a micron-sized silica and from about 7.5 up to about 8.0 mg sodium stearyl fumarate.

In still another embodiment, in the inner core

the granulated admixture comprises above 0 mg sodium lauryl sulfate, about 45 mg of a microcrystalline cellulose, about 7 mg of a hydroxypropylcellulose, and about 22 mg carbidopa;

about 114 mg levodopa ethyl ester is present; and

the inner core excipient component comprises about 35 mg of a hydroxypropylmethylcellulose, about 25 mg of a microcrystalline cellulose, about 3 mg of a micron-sized silica and about 5 mg of sodium stearyl fumarate,

and wherein in the outer layer

about 32 mg granulated carbidopa is present in a granulated admixture with about 24 mg of a microcrystalline cellulose, about 20 mg of a partially pregelatinized maize starch, and about 7 mg of a hydroxypropylcellulose.

The subject invention further provides a tablet which comprises

an inner core formulated for controlled release comprising a mixture of

(a) a granulated admixture of above 0 mg up to about 100 mg carbidopa, from about 0.03 mg to about 75 mg of a surfactant and at least one excipient,

(b) from about 4 mg up to about 400 mg levodopa ethyl ester or a derivative or a pharmaceutically acceptable salt thereof, and

(c) an inner core excipient component;

and an outer layer encapsulating the inner core and formulated for immediate release comprising a mixture of

(i) a granulated admixture of from about 1 mg up to about 75 mg carbidopa and at least one excipient; and

(ii) from about 5 mg up to about 300 mg levodopa ethyl ester or a derivative or a pharmaceutically acceptable salt thereof, and

(iii) an outer layer excipient component.

In one embodiment, the surfactant is an ionic surfactant. In another embodiment, the surfactant is an aionic surfactant.

The subject invention also provides a process for manufacturing the tablet of the subject invention, comprising

(A) preparing a granulated admixture of a decarboxylase inhibitor and a surfactant;

(B) mixing the granulated admixture from of step (A) with levodopa ethyl ester or a derivative or a pharmaceutically acceptable salt thereof;

(C) compressing the mixture from step (B) to form the inner core;

(D) separately mixing a granulated decarboxylase inhibitor with levodopa ethyl ester or a derivative or a pharmaceutically acceptable salt thereof;

(E) compressing the mixture of step (D) over the inner core formed in step (C) to form an outer layer encapsulating the inner core so as to thereby manufacture the tablet.

In one embodiment, the process for manufacturing the tablet of the subject invention comprises

(A) preparing a granulated admixture of carbidopa and a surfactant;

(B) mixing the granulated admixture from step (A) with an inner core excipient and levodopa ethyl ester or a derivative or a pharmaceutically acceptable salt thereof;

(C) compressing the mixture from step (B) to form the inner core;

(D) separately mixing carbidopa with an outer layer excipient and levodopa ethyl ester or a derivative or a pharmaceutically acceptable salt thereof;

(E) compressing the mixture of step (D) over the inner core formed in step (C) to form an outer layer encapsulating the inner core so as to thereby manufacture the tablet.

In another embodiment, the process for manufacturing the tablet of the subject invention comprises

(A) preparing a granulated admixture of carbidopa and a surfactant;

(B) mixing the granulated admixture from of step (A) with an inner core excipient component and levodopa ethyl ester or a derivative or a pharmaceutically acceptable salt thereof;

(C) compressing the mixture from step (B) to form the inner core;

(D) separately mixing granulated carbidopa with an outer layer excipient and levodopa ethyl ester or a derivative or a pharmaceutically acceptable salt thereof;

(E) compressing the mixture of step (D) over the inner core formed in step (C) to form an outer layer encapsulating the inner core so as to thereby manufacture the tablet.

In a further embodiment, in step (B) and/or in step (D), the levodopa ethyl ester or derivative or pharmaceutically acceptable salt thereof are present in a ratio by weight of carbidopa to levodopa ethyl ester from about 0.01:1 up to about 1:1.

In yet another embodiment, the process for manufacturing the tablet of the subject invention, comprises

(A) preparing a granulated admixture of above 0 mg up to about 100 mg carbidopa and from about 0.03 mg to about 75 mg of a surfactant and at least one excipient;

(B) mixing the granulated admixture from of step (A) with from about 4 mg up to about 400 mg levodopa ethyl ester or a derivative or a pharmaceutically acceptable salt thereof and an inner core excipient component;

(C) compressing the mixture from step (B) to form the inner core;

(D) separately granulating from about 1 mg to about 75 mg carbidopa with at least one excipient to form a granulated admixture;

(E) mixing the granulated admixture from step (D) with an outer core excipient and from about 5 mg up to about 300 mg levodopa ethyl ester or a derivative or a pharmaceutically acceptable salt thereof;

(F) compressing the mixture of step (E) over the inner core formed in step (C) to form an outer layer encapsulating the inner core so as to thereby manufacture the tablet.

The subject invention additionally provides a method of treating a subject suffering from a disease selected from the group consisting of Parkinson's disease, senile dementia, dementia of the Alzheimer's type, a memory disorder, depression, hyperactive syndrome, an affective illness, a neurodegenerative disease, a neurotoxic injury, brain ischemia, a head trauma injury, a spinal trauma injury, schizophrenia, an attention deficit disorder, multiple sclerosis, withdrawal symptoms, epilepsy, convulsions and seizures, which comprises administering to the subject the tablet of the subject invention in an amount effective to treat the disease. In a preferred embodiment, the disease is Parkinson's disease. In one embodiment of the subject invention, the treatment of Parkinsonian patients is long-term. The therapeutically effective amount of LDEE is preferably an amount from 0.1-1000 mg equivalent of levodopa.

The external core of the tablet comprises the fast onset “burst” immediate-release formulation. The internal core comprises a controlled or slow-release (up to 3-8 hours) formulation using an approved cellulose derivative which swells and/or becomes gellable and erodible on contact with water or aqueous solutions.

As discussed in the background, levodopa is often administered with a decarboxylase inhibitor. In a solid formulation, it is important that the rate of dissolution, and hence, the blood level, of the decarboxylase inhibitor be appropriate for that of the L-DOPA. In both the immediate release and the slow release formulations of carbidopa and L-DOPA, these two active ingredients are released at the same ratio. This release can be readily achieved in a matrix system because the chemical and physical properties of carbidopa and levodopa are similar. In monolithic matrix systems, the active agents are homogeneously dissolved or dispersed throughout a polymer mass or other carrier material. Release characteristics depend on the geometry of the system, the nature of the polymer and other excipients, solubility and the processing methods. As the two active materials, carbidopa and L-DOPA, were compressed under the same conditions, it was found that the in vitro release is a direct function of their solubility in the dissolution fluid. Both compounds are slightly soluble in water, degrade rapidly in alkaline media, and have similar solubility versus pH profiles, as well as similar solubility versus temperature profiles.

However, carbidopa and LDEE, examples of two active materials in the composition of the present invention, have different chemical and physical properties and contrasting solubility characteristics especially in acid conditions, i.e., in the gastrointestinal tract (see Table 1).

TABLE 1

| | Solubility at ˜22-24° C. (mg/ml) | | |
|---|---|---|---|
| pH | Levodopa | Carbidopa | LDEE |
| 1.0 | 5.38 | 21.4 | 2200 |
| 5.0 | 4.96 | 1.66 | 1700 |

Formulating a pharmaceutical composition from LDEE and carbidopa, examples of two active ingredients of the subject invention having different solubility characteristics, which were to be released with similar immediate and controlled release dissolution profiles from a dual release tablet presented a problem. In detail,

(a) Carbidopa contains 7.5% water whereas LDEE is highly sensitive to moisture, undergoes hydrolysis easily, and therefore would not be expected to be a candidate for a slow release formulation;

(b) LDEE has a tendency to act as a binder (it sticks).

(c) LDEE is not stable at room temperature and is kept under refrigeration (2-8° C.), whereas any tablet formulation must be designed, for optimum convenience to patients, pharmacists and physicians, for storage at room temperature.

The subject invention has overcome these difficulties. To attain a similar rate of release for levodopa ethyl ester and carbidopa, the disclosed formulation uses levodopa ethyl ester and carbidopa in both the immediate release and the controlled release portions of the tablet. Thus, as shown in Table 15, the disclosed formulation exhibits approximately equal release profiles of carbidopa and levodopa ethyl ester in the immediate release portion. Table 7, especially Inner Core D, reflects the substantially similar release profiles of carbidopa and levodopa ethyl ester in the inner core. Carbidopa has a significantly higher half-life and lower solubility in the body than levodopa, so the immediate release carbidopa is present in the body longer than the levodopa metabolized from the immediate release levodopa ethyl ester. While the level of carbidopa from the immediate release portion is decreasing, the overall level of carbidopa and the overall level of levodopa ethyl ester are maintained at substantially the same level by the controlled release layer. Thus, the disclosed tablet formulation provides therapeutically appropriate levels of levodopa ethyl ester and carbidopa in vivo.

As discussed in the background of the invention, Rubin (U.S. Pat. No. 6,238,699 B1) describes a formulation of carbidopa and levodopa in immediate and controlled release compartments. However, Rubin does not suggest the replacement of levodopa with LDEE or how to account for the difference in properties between the LDEE and levodopa. Given the differences in bio-availability and solubility described above, one skilled in the art would not be motivated to replace levodopa with LDEE to create a pharmaceutical composition containing LDEE and carbidopa in immediate and controlled release portions of a tablet as in the subject invention.

Chiesi et al. (WO 99/17745), also described in the background of the invention, disclose a three-layer monolithic system of levodopa methyl ester and carbidopa. Chiesi et al. show that the release of levodopa methyl ester is approximately concomitant with the release of carbidopa in a formulation in which levodopa methyl ester, but not carbidopa, is present in the slow release layer, while both levodopa methyl ester and carbidopa are used in the remaining layers. Additionally, the release profile of the levodopa methyl ester and the carbidopa in the compositions of Chiesi et al. is dependent upon the interactions between the layers of the composition.

In contrast to Chiesi et al., due to the different geometry of the pharmaceutical compositions of the subject invention, the release profiles of the LDEE and the carbidopa are independent. In the subject invention, the total release kinetic is the sum of the individual contributions and one can predict from the dissolution profile of the inner core the total release kinetics of the dual release tablet. Furthermore, the metabolic products of levodopa methyl ester are levodopa and methanol (which is toxic, U.S. Pat. No. 5,354,885), while the metabolic products of LDEE are levodopa and ethanol. In addition, the tablet of the subject invention differs from Chiesi et al. in that the subject invention is easier to manufacture, is physically smaller, and is therefore expected to have higher patient compliance.

The LDEE used in the compositions of the present invention is preferably that as described in U.S. Pat. Nos. 6,218,566 or 5,525,631, the contents of which are hereby incorporated by reference. LDEE may be prepared following the procedure of U.S. Pat. Nos. 6,218,566; 5,607,969; 5,525,631; or 5,354,885; all of which are hereby incorporated by reference. Preferably, the LDEE is highly purified, stable, non-hygroscopic and crystalline.

The subject invention provides press-coated tablets, multi-layered tablets and a combination of a matrix and a disintegrant tablet.

This invention will be better understood from the Experimental Details which follow. However, one skilled in the art will readily appreciate that the specific methods and results discussed are merely illustrative of the invention as described more fully in the claims which follow thereafter.

Experimental Details

LDEE was prepared as described in U.S. Pat. No. 6,218,566 B1. However, any pharmaceutically acceptable salt of LDEE can be used.

### EXAMPLE 1

### Manufacture of LDEE Tablets

Inner Core

Inner core A (controlled release) was manufactured as follows:

Procedure 1

Carbidopa monohydrate was granulated with a wetting agent and several excipients to form a carbidopa monohydrate granulate (Table 2).

TABLE 2
Composition of carbidopa monohydrate granulate in
controlled release inner core

| Excipient | Use | A (mg/tablet) |
|---|---|---|
| Carbidopa | Active material | 27 |
| monohydrate | | |
| Sodium lauryl | Wetting agent | 1 |
| Sulphate | | |
| Microcrystalline | Filler | 56 |
| cellulose | | |
| Hydroxypropyl- | Binding agent | 9 |
| cellulose | | |

The carbidopa monohydrate granulate was then mixed with LDEE, a carrier and several excipients (Table 3).

TABLE 3
Composition of inner core - controlled release

| Excipient | Use | A (mg/tablet) |
|---|---|---|
| Granulate | Active | 93 |
| carbidopa | material | |
| monohydrate (see | | |
| Table 2) | | |
| L-Dopa ethyl ester | Active material | 114 |
| Hydroxypropyl- | Carrier | 35 |
| methylcellulose | | |
| Microcrystalline | Filler | 25 |
| cellulose | | |
| Syloid ® | Flow agent | 3 |
| Sodium stearyl | Lubricant | 5 |
| fumarate | | |
| Magnesium stearate | Lubricant | — |

The resulting mixture was compressed into inner core A.

Outer Layer

The outer layer, the immediate release formulation, was prepared as described in Procedure 1 above using the components in the amounts listed in Tables 4 and 5 was compressed onto inner core A to produce press-coated tablet A.

TABLE 4
Composition of carbidopa monohydrate
granulate for outer layer

| Excipient | Use | A (mg/tablet) |
|---|---|---|
| Carbidopa | Active material | 27 |
| monohydrate | | |
| Microcrystalline | Filler | 20 |
| cellulose | | |
| Starch 1500 ® | Disintegrating | 17 |
| | agent | |
| Hydroxypropyl- | Binding agent | 6 |
| cellulose | | |

TABLE 5
Composition of outer layer - immediate release

| Excipient | Use | A (mg/tablet) |
|---|---|---|
| Granulate | Active material | 70 |
| carbidopa | | |
| monohydrate (see | | |
| Table 4) | | |
| L-Dopa ethyl ester | Active material | 114 |
| Microcrystalline | Filler | 80 |
| cellulose | | |
| Starch 1500 ® | Disintegrating | 4 |
| | agent | |
| Syloid ® | Flow agent | 3 |
| Sodium stearyl | Lubricant | 8 |
| fumerate | | |

### EXAMPLE 2

Each of the following inner cores C-F (prepared according to Procedure 1) contained a different amount of the wetting agent sodium lauryl sulfate in order to try to achieve the same dissolution rate for carbidopa monohydrate and LDEE (Table 6).

TABLE 6
Variation in the amount of wetting agent

| Inner | | | C | D | E | F |
|---|---|---|---|---|---|---|
| core | Excipient | Use | mg/tab | mg/tab | mg/tab | mg/tab |
| Granulate | Carbidopa | Active | 27 | 27 | 27 | 27 |
| | monohydrate | material | | | | |
| | Sodium | Wetting | 0 | 0.2 | 1 | 5 |
| | lauryl | agent | | | | |
| | sulfate | | | | | |
| | Microcrystalline | Filler | 56 | 56 | 56 | 56 |
| | cellulose | | | | | |
| | Hydroxypropyl-cellulose | Binding | 9 | 9 | 9 | 9 |
| | | agent | | | | |
| | L-Dopa ethyl | Active | 114 | 114 | 114 | 114 |
| | ester | material | | | | |
| | Hydroxypropyl-methylcellulose | Carrier | 35 | 35 | 35 | 35 |
| | Microcrystalline | Filler | 25 | 25 | 25 | 25 |
| | cellulose | | | | | |
| | Syloid ® | Flow | 3 | 3 | 3 | 3 |
| | | agent | | | | |
| | Sodium stearyl | Lubricant | 5 | 5 | 5 | 5 |
| | fumarate | | | | | |
| | Magnesium | Lubricant | — | — | — | — |
| | stearate | | | | | |

Each inner core formulation was then tested in a dissolution test using 900 ml 0.1 N HCl at 37° C. in US Pharmacopeia (USP) Apparatus 2 at 75 RPM.

The release profile for each inner core over a 3 hour period is shown in Table 7 below.

TABLE 7
Dissolution of carbidopa monohydrate/LDEE of inner cores C-F

| | C | | D | | E | | F | |
|---|---|---|---|---|---|---|---|---|
| | | % Release | | % Release | | % Release | | % Release |
| Time | % Release | carbidopa | % Release | carbidopa | % Release | carbidopa | % Release | carbidopa |
| (hour) | LDEE | monohydrate | LDEE | monohydrate | LDEE | monohydrate | LDEE | monohydrate |
| 0.5 | 40 | 24 | 54 | 54 | 38 | 30 | 31 | 21 |
| 1 | 54 | 36 | 68 | 67 | 54 | 46 | 44 | 34 |
| 1.5 | 65 | 45 | 74 | 74 | 65 | 59 | 54 | 45 |
| 2 | 74 | 53 | 79 | 79 | 75 | 72 | 61 | 53 |
| 2.5 | 79 | 60 | 81 | 82 | 82 | 82 | 67 | 59 |
| 3 | 85 | 67 | 83 | 85 | 88 | 92 | 72 | 66 |

The release of carbidopa monohydrate and LDEE from the inner core was more concomitant when using the wetting agent sodium lauryl sulphate (inner cores D-F) as compared to not using such a wetting agent (inner core C). Optimal results were obtained with 0.2 mg and 1 mg/tablet wetting agent (inner cores D and E): the difference between the dissolution rate of LDEE and carbidopa monohydrate was reduced as compared to the other amounts of wetting agent tested.

### EXAMPLE 3

Each of the following inner cores G-J (prepared according to Procedure 1) contained a different amount of the carrier hydroxypropylmethylcellulose (brand name Methocel® K100LV) in order to determine the effect of the amount of the carrier on the dissolution rate (see Table 8).

TABLE 8
Variation in the amount of carrier

| Inner | | | G | H | I | J |
|---|---|---|---|---|---|---|
| core | Excipient | Use | mg/tab | mg/tab | mg/tab | mg/tab |
| Granulate | Carbidopa | Active | 27 | 27 | 27 | 27 |
| | monohydrate | material | | | | |
| | Sodium lauryl | Wetting | 1 | 1 | 1 | 1 |
| | sulphate | agent | | | | |
| | Microcrystalline | Filler | 56 | 56 | 56 | 56 |
| | cellulose | | | | | |
| | Hydroxypropyl-cellulose | Binding | 9 | 9 | 9 | 9 |
| | | agent | | | | |
| | L-Dopa ethyl | Active | 114 | 114 | 114 | 114 |
| | ester | material | | | | |
| | Hydroxypropyl-methylcellulose | Carrier | 70 | 40 | 30 | 20 |
| | Microcrystalline | Filler | 25 | 25 | 25 | 25 |
| | cellulose | | | | | |
| | Syloid ® | Flow | 3 | 3 | 3 | 3 |
| | agent | | | | | |
| | Sodium stearyl | Lubricant | 5 | 5 | 5 | 5 |
| | fumarate | | | | | |
| | Magnesium | Lubricant | — | — | — | — |
| | stearate | | | | | |

The inner cores were then subjected to a dissolution test as in Example 2. The release profile for each inner core formulation over an 8 hour period is shown in Table 9.

TABLE 9
Dissolution of carbidopa monohydrate/LDEE of inner cores G-J

| | G | | H | | I | | J | |
|---|---|---|---|---|---|---|---|---|
| | | % Release | | % Release | | % Release | | % Release |
| Time | % Release | carbidopa | % Release | carbidopa | % Release | carbidopa | % Release | carbidopa |
| (hour) | LDEE | monohydrate | LDEE | monohydrate | LDEE | monohydrate | LDEE | monohydrate |
| 0.5 | 25 | 14 | 33 | 15 | 40 | 22 | 56 | 47 |
| 1 | 38 | 24 | 47 | 25 | 57 | 36 | 75 | 63 |
| 1.5 | — | — | 56 | 34 | 69 | 48 | 85 | 72 |
| 2 | 57 | 45 | 67 | 43 | 78 | 59 | 92 | 79 |
| 2.5 | — | — | 75 | 52 | 86 | 70 | 97 | 84 |
| 3 | — | — | 83 | 64 | 92 | 79 | 101 | 89 |
| 6 | 93 | 97 | — | — | — | — | — | — |
| 8 | 96 | 102 | — | — | — | — | — | — |

The optimal results for dissolution of 3-4 hours were obtained when using 30-40 mg/tab of the carrier (Methocel® K100LV) in inner cores I and H. When using 20 mg/tab of the carrier (Methocel® K100LV) in inner core J, a full release was obtained after 2 hours. For a dissolution profile of 8 hours, 70 mg/tab of the carrier (inner core G) was optimal.

### EXAMPLE 4

Each one of inner cores N and P (produced according to procedure 1) contained a different carrier as described in Table 10 in order to determine the effect of the carrier on the dissolution rate shown in Table 11.

TABLE 10
Variation in the carrier

| Inner core | Excipient | Use | N mg/tab | P mg/tab |
|---|---|---|---|---|
| Granulate | Carbidopa | Active | 27 | 27 |
| | monohydrate | material | | |
| | Sodium lauryl | Wetting | 1.0 | 1.0 |
| | Sulphate | agent | | |
| | Microcrystalline | Filler | 56 | 56 |
| | cellulose | | | |
| | Hydroxypropyl- | Binding | 9 | 9 |
| | cellulose | agent | | |
| | L-Dopa ethyl ester | Active | 114 | 114 |
| | | material | | |
| | Methocel K100LV ® | Carrier | — | 35 |
| | Methocel K15M ® | Carrier | 35 | — |
| | Microcrystalline | Filler | 25 | 25 |
| | cellulose | | | |
| | Syloid ® | Flow agent | 3 | 3 |
| | Sodium stearyl | Lubricant | 5 | 5 |
| | fumarate | | | |
| | Magnesium stearate | Lubricant | — | — |

The dissolution tests were performed as described in Example 2.

TABLE 11
Dissolution of LDEE/carbidopa
monhydrate of inner cores N and P

| | N | | P | |
|---|---|---|---|---|
| | | % Release | | % Release |
| Time | % Release | carbidopa | % Release | carbiadopa |
| (hour) | LDEE | monohydrate | LDEE | monohydrate |
| 0.5 | 57 | 37 | 38 | 30 |
| 1 | 66 | 49 | 54 | 46 |
| 1.5 | 75 | 56 | 65 | 59 |
| 2 | 82 | 62 | 75 | 72 |
| 2.5 | 87 | 68 | 82 | 82 |
| 3 | 91 | 72 | 88 | 92 |

The carrier Methocel® K100LV gave slower dissolution results compared to the carrier Methocel® K15M with the same amount used. Both of the carriers were useful to obtain a desired dissolution profile.

### EXAMPLE 5

Each of the inner cores contained different amounts of carbidopa monohydrate granulate as described in Table 12 in order to see if the dissolution is affected by the amount of the carbidopa monohydrate granulate. Inner cores S, T, and U were prepared according to Procedure 1. Inner core V was prepared according to Procedure 2: The inner core was prepared by mixing LDEE with a carrier and several excipients.

TABLE 12
Variation in amounts of carbidopa monohydrate in inner core

| | | | S | | | |
|---|---|---|---|---|---|---|
| | | | 100%¹ | T 90% | U 80% | V 0% |
| | Excipient | Use | (mg/tab) | (mg/tab) | (mg/tab) | (mg/tab)* |
| Granulate | Carbidopa | Active | 27 | 24 | 22 | — |
| | monohydrate | material | | | | |
| | Sodium lauryl | Wetting | 1 | 0.9 | 0.8 | — |
| | sulphate | agent | | | | |
| | Microcrystal-line | Filler | 56 | 50 | 45 | — |
| | cellulose | | | | | |
| | Hydroxypropyl-cellulose | Binding | 9 | 8 | 7 | — |
| | | agent | | | | |
| Inner | Granulate | Active | 93 | 83 | 75 | 0 |
| core | carbidopa | material | | | | |
| | monohydrate | | | | | |
| | L-Dopa ethyl | Active | 114 | 114 | 114 | 114 |
| | ester | material | | | | |
| | Hydroxypropyl- | Carrier | 35 | 35 | 35 | 70 |
| | methyl- | | | | | |
| | cellulose | | | | | |
| | Microcrystal-line | Filler | 25 | 25 | 25 | 85 |
| | cellulose | | | | | |
| | Syloid ® | Flow agent | 3 | 3 | 3 | 3 |
| | Sodium stearyl | Lubricant | 5 | 5 | 5 | 5 |
| | fumarate | | | | | |
| | Magnesium | Lubricant | — | — | — | 3 |
| | stearate | | | | | |

¹Mg of carbidopa monohydrate relative to mg of carbidopa monohydrate in inner core S

The dissolution test was performed as described in Example 2. The release profile is shown in Table 13 below.

TABLE 13
Dissolution or LDEE/carbidopa monohydrate of inner cores S-V

| | S | | T | | U | | V | |
|---|---|---|---|---|---|---|---|---|
| | | % Release | | % Release | | % Release | | % Release |
| Time | % Release | carbidopa | % Release | carbidopa | % Release | carbidopa | % Release | carbidopa |
| (hour) | LDEE | monohydrate | LDEE | monohydrate | LDEE | monohydrate | LDEE | monohydrate |
| 0.5 | 38 | 30 | 29 | 21 | 34 | 28 | 31 | — |
| 1 | 54 | 46 | 43 | 37 | 46 | 39 | 47 | — |
| 1.5 | 65 | 59 | 54 | 50 | 56 | 49 | 58 | — |
| 2 | 75 | 72 | 62 | 62 | 68 | 62 | 62 | — |
| 2.5 | 82 | 82 | 70 | 72 | 75 | 71 | 75 | — |
| 3 | 88 | 92 | 76 | 81 | 81 | 78 | 83 | — |

* Inner core S is identical to inner core A (described in Example 1).

Even though the amounts of carbidopa monohydrate in the inner core granulate were varied, there were no significant differences in dissolution profile nor physical properties.

### EXAMPLE 6

The following tablet was prepared, accoring to Procedure 1, containing an immediate release formulation of LDEE and carbidopa monohydrate (“outer layer” of the subject invention without the “inner core”) (Table 14).

TABLE 14
Outer layer

| | Excipient | Use | mg/tablet |
|---|---|---|---|
| Carbidopa | Carbidopa | Active | 32 |
| monohydrate | monohydrate | material | |
| granulate | Microcrystalline | Filler | 24 |
| | cellulose | | |
| | Starch 1500 ® | Disintegrating | 20.4 |
| | | agent | |
| | Hydroxpropyl- | Binding agent | 7.2 |
| | cellulose | | |
| | L-DOPA ethyl | Active | 114 |
| | ester | material | |
| | Microcrystalline | Filler | 80 |
| | cellulose | | |
| | Starch 1500 ® | Disintegrating | 4 |
| | | agent | |
| | Syloid ® | Flow agent | 3 |
| | Sodium stearyl | Lubricant | 7.5 |
| | fumarate | | |

Each tablet was tested in a dissolution bath as in Example 2. The dissolution profile is presented in Table 15.

TABLE 15
Dissolution of LDEE/carbidopa
monohydrate from outer layer

| | carbidopa | |
|---|---|---|
| Time (minutes) | monohydrate | LDEE |
| 5 | 103 | 94 |
| 10 | 104 | 95 |
| 15 | 104 | 94 |

The entire amount of the 2 active materials in the outer layer were released after 5 minutes. The inherent solubility of the 2 active ingredients (LDEE and carbidopa) gives rapid release in this formulation.

### EXAMPLE 7

The following tablet W was prepared (Table 16). The inner core and the outer layer were manufactured according to Procedure 1.

TABLE 16
Dual release tablet composition W

| Portion of | | | | W |
|---|---|---|---|---|
| Tablet | | Excipient | Use | (mg/tab) |
| Inner | Granulate | Carbidopa monohydrate | Active material | 22 |
| core | | Sodium lauryl | Wetting agent | 0.8 |
| | | sulphate | | |
| | | Microcrystalline | Filler | 45 |
| | | cellulose | | |
| | | Hydroxypropyl- | Binding agent | 7 |
| | | cellulose | | |
| | | L-Dopa ethyl ester | Active material | 114 |
| | | Hydroxypropylmethyl- | Carrier | 35 |
| | | cellulose | | |
| | | Microcrystalline | Filler | 25 |
| | | cellulose | | |
| | | Syloid ® | Flow agent | 3 |
| | | Sodium stearyl fumarate | Lubricant | 5 |
| | | Magnesium stearate | Lubricant | — |
| | | Excipient | Use | |
| Outer | Granulate | Carbidopa monohydrate | Active material | 32 |
| layer | | Microcrystalline | Filler | 24 |
| | | cellulose | | |
| | | Starch 1500 ® | Disintegrating | 20 |
| | | | agent | |
| | | Hydroxypropylcellulose | Binding agent | 7 |
| | | L-Dopa ethyl ester | Active material | 114 |
| | | Microcrystalline | Filler | 80 |
| | | cellulose | | |
| | | Starch 1500 ® | Disintegrating | 4 |
| | | | agent | |
| | | Syloid ® | Flow agent | 3 |
| | | Sodium stearyl fumarate | Lubricant | 8 |

The release profile is shown in Table 17 below.

TABLE 17
Dissolution of LDEE/carbidopa monohydrate in tablet W

W

| | | % Release |
|---|---|---|
| | % Release L-Dopa | carbidopa |
| Time (min) | ethyl ester | monohydrate |
| 5 | 55 | 66 |
| 10 | 61 | 71 |
| 30 | 71 | 78 |
| 60 | 79 | 83 |
| 90 | 84 | 89 |
| 120 | 88 | 92 |
| 150 | 91 | 96 |
| 180 | 94 | 99 |

In the LDEE/carbidopa monohydrate dissolution profile of Tablet W, half of the amount of the two active materials was released in the first 5 minutes and the rest was controlled released thereafter.

### EXAMPLE 7

Each of the following inner cores prepared according to Procedure 1 contained a different lot of LDEE in order to determine the effect of the particle size on the dissolution (see Tables 18-19).

TABLE 18
Particle size distribution of
LDEE in inner cores Y and Z

| | Y (μ) | Z (μ) |
|---|---|---|
| d(0.1) | 14 | 71 |
| d(0.5) | 67 | 46.5 |
| d(0.9) | 222 | 122 |

TABLE 19
Composition of inner cores Y and Z

| Inner | | | Y | Z |
|---|---|---|---|---|
| core | Excipient | Use | (mg/tab) | (mg/tab) |
| Granulate | Carbidopa | Active | 22 | 24 |
| | monohydrate | material | | |
| | Sodium lauryl | Wetting | 1 | 1 |
| | sulphate | agent | | |
| | Microcrystalline | Filler | 45 | 50 |
| | cellulose | | | |
| | Hydroxypropyl- | Binding | 7 | 7 |
| | cellulose | agent | | |
| | L-Dopa ethyl ester | Active | 114 | 114 |
| | | material | | |
| | Hydroxypropyl- | Carrier | 35 | 35 |
| | methycellulose | | | |
| | Microcrystalline | Filler | 25 | 25 |
| | cellulose | | | |
| | Syloid ® | Flow agent | 3 | 3 |
| | Sodium stearyl | Lubricant | 5 | 5 |
| | fumarate | | | |
| | Magnesium stearate | Lubricant | — | — |

The dissolution profiles, determined as in Example 2, are presented in Table 20.

TABLE 20
Dissolution of LDEE/carbidopa
monohydrate of inner cores Y and Z

| | Y | | Z | |
|---|---|---|---|---|
| | | % Release | | % Release |
| Time | | carbidopa | % Release | carbidopa |
| (hr) | % Release LDEE | monohydrate | LDEE | monohydrate |
| 0.5 | 34 | 28 | 29 | 21 |
| 1 | 46 | 39 | 43 | 37 |
| 1.5 | 56 | 49 | 54 | 50 |
| 2 | 68 | 62 | 62 | 62 |
| 2.5 | 75 | 71 | 70 | 72 |
| 3 | 81 | 78 | 76 | 81 |

No significant differences were obtained when using a different lot or different particle size of LDEE (see Table 20).

What is claimed is:

1. A tablet which comprises:
an inner core formulated for controlled release comprising a mixture of
(a) a granulated admixture of a decarboxylase inhibitor and a surfactant, and
(b) levodopa ethyl ester or a derivative or a pharmaceutically acceptable salt thereof; and
an outer layer encapsulating the inner core and formulated for immediate release comprising a mixture of granulated decarboxylase inhibitor and levodopa ethyl ester or a derivative or a pharmaceutically acceptable salt thereof.

2. The tablet of claim 1, wherein the surfactant is an ionic or an aionic surfactant.

3. A tablet which comprises:
an inner core formulated for controlled release comprising a mixture of
(a) a granulated admixture of from above 0 mg up to about 100 mg carbidopa, and from about 0.03 mg to about 75 mg surfactant,
(b) from about 4 mg up to about 400 mg levodopa ethyl ester or a derivative or a pharmaceutically acceptable salt thereof, and
(c) an inner core excipient component; and
an outer layer encapsulating the inner core and formulated for immediate release comprising a mixture of:
(i) from above 0 mg up to about 75 mg carbidopa,
(ii) from about 5 mg up to about 300 mg levodopa ethyl ester or a derivative or a pharmaceutically acceptable salt thereof, and
(iii) an outer layer excipient component.

4. The tablet of claim 3, wherein the surfactant is an ionic or an aionic surfactant.

5. The tablet of claim 3, wherein in the outer layer, the carbidopa in (i) comprises granulated carbidopa.

6. The tablet of any one of claims 3 to 5, wherein the inner core is formulated such that the rate of release of the carbidopa is substantially the same as the rate of release of the levodopa ethyl ester or derivative or pharmaceutically acceptable salt thereof.

7. The tablet of any one of claims 3 to 5, wherein the outer layer is formulated such that the rate of release of the carbidopa is substantially the same as the rate of release of the levodopa ethyl ester or derivative or pharmaceutically acceptable salt thereof.

8. The tablet of claim 7, wherein the inner core is formulated such that the rate of release of the carbidopa is substantially the same as the rate of release of the levodopa ethyl ester or derivative or pharmaceutically acceptable salt thereof.

9. The tablet of any one of claims 3 to 5, wherein the inner core excipient component comprises an excipient used as a carrier.

10. The tablet of claim 9, wherein the excipient used as a carrier comprises a hydroxypropylmethylcellulose.

11. The tablet of claim 10, wherein the hydroxypropylmethylcellulose has an average molecular weight between about 10 kDa and about 1500 kDa.

12. The tablet of claim 11, wherein the hydroxypropylmethylcellulose has 19%-24% methoxyl substituent and 7%-12% hydroxylproproxyl substituent.

13. The tablet of claim 12, wherein the hydroxypropylmethylcellulose has a particle size distribution such that 65%-85% of the hydroxypropylmethylcellulose passes through a 100 mesh screen.

14. The tablet of claim 13, wherein the hydroxypropylmethylcellulose has a particle size distribution such that about 80% of the hydroxypropylmethylcellulose passes through a 100 mesh screen.

15. The tablet of any one of claims 3 to 5, wherein the outer layer excipient component comprises an excipient used as a binding agent.

16. The tablet of claim 15, wherein the excipient used as a binding agent comprises a hydroxypropylcellulose.

17. The tablet of any one of claims 3 to 5, wherein the outer layer excipient component comprises an excipient used as a disintegrating agent.

18. The tablet of claim 17, wherein the excipient used as a disintegrating agent comprises a starch.

19. The tablet of claim 18, wherein the starch is a partially pregelatinized maize starch.

20. The tablet of any one of claims 3 to 5, wherein the inner core excipient component and the outer layer excipient component each comprise an excipient useful as a flow agent and/or an excipient useful as a lubricant.

21. The tablet of claim 20, wherein the excipient useful as a flow agent comprises a micron-sized silica powder.

22. The tablet of claim 20, wherein the excipient useful as a lubricant comprises magnesium stearate.

23. The tablet of claim 20, wherein the excipient useful as a lubricant comprises sodium stearyl fumarate.

24. The tablet of any one of claims 3 to 5, wherein the inner core excipient component and the outer layer excipient component each comprise an excipient useful as a lubricant.

25. The tablet of any one of claims 3 to 5, wherein the same excipient useful as a lubricant is present in both the inner core excipient component and the outer layer excipient component.

26. The tablet of claim 24 or 25, wherein the excipient useful as a lubricant present in the outer layer excipient component comprises sodium stearyl fumarate.

27. The tablet of claim 24, wherein the excipient useful as a lubricant present in the inner core excipient component comprises sodium stearyl fumarate.

28. The tablet of claim 20, wherein the inner core excipient component comprises a first excipient useful as a lubricant and a second excipient useful as a lubricant.

29. The tablet of claim 28, wherein the first excipient useful as a lubricant is sodium stearyl fumarate and the second excipient useful as a lubricant is magnesium stearate.

30. The tablet of any one of claims 3 to 5, wherein the inner core excipient component and/or the outer layer excipient component comprises an excipient useful as a filler.

31. The tablet of claim 30, wherein the excipient useful as a filler comprises a microcrystalline cellulose.

32. The tablet of claim 31, wherein the microcrystalline cellulose has an average particle size between about 50 and about 90 microns.

33. The tablet of claim 4, wherein in the inner core
the granulated admixture in (a) comprises from above 0 mg up to about 75 mg carbidopa, and from about 0.03 mg up to about 50 mg ionic surfactant,
the levodopa ethyl ester or derivative or pharmaceutically acceptable salt thereof in (b) is present in an amount from about 10 mg up to about 400 mg;
and wherein in the outer layer,
the granulated carbidopa in (i) comprises from above 0 mg to about 75 mg carbidopa, and
the levodopa ethyl ester or derivative or pharmaceutically acceptable salt thereof in (ii) is present in an amount from about 10 mg up to about 250 mg.

34. The tablet of claim 33, wherein in the inner core
the ionic surfactant is present in an amount from about 0.1 mg up to about 50 mg, and
the levodopa ethyl ester or derivative or pharmaceutically acceptable salt thereof is present in an amount from about 50 mg up to about 400 mg;
and wherein in the outer layer
the granulated carbidopa comprises from about 10 mg up to about 50 mg carbidopa, and
the amount of the levodopa ethyl ester or the derivative or pharmaceutically acceptable salt thereof is from about 50 mg up to about 200 mg.

35. The tablet of claim 33, wherein in the inner core
the granulated admixture comprises from 4.2 mg up to about 50 mg carbidopa and from about 0.1 mg up to about 10 mg ionic surfactant, and
the levodopa ethyl ester or derivative or pharmaceutically acceptable salt thereof is present in an amount from about 19 mg up to about 228 mg;
and wherein in the outer layer
the granulated carbidopa comprises from about 4.2 mg up to about 50 mg carbidopa, and
the levodopa ethyl ester or derivative or pharmaceutically acceptable salt thereof is present in an amount from about 19 mg up to about 228 mg.

36. The tablet of any one of claims 3 to 5, wherein above 5% of the total levodopa ethyl ester or derivative or pharmaceutically acceptable salt thereof present in the tablet is in the outer layer.

37. The tablet of claim 36, wherein above 10% of the total levodopa ethyl ester or derivative or pharmaceutically acceptable salt thereof present in the tablet is in the outer layer.

38. The tablet of claim 37, wherein above 30% of the total levodopa ethyl ester or derivative or pharmaceutically acceptable salt thereof present in the tablet is in the outer layer.

39. The tablet of claim 38, wherein above 50% of the total levodopa ethyl ester or derivative or pharmaceutically acceptable salt thereof present in the tablet is in the outer layer.

40. The tablet of claim 39, wherein above 70% of the total levodopa ethyl ester or derivative or pharmaceutically acceptable salt thereof present in the tablet is in the outer layer.

41. The tablet of any one of claims 3 to 5, wherein above 10% of the total carbidopa present in the tablet is in the outer layer.

42. The tablet of claim 41, wherein above 30% of the total carbidopa present in the tablet is in the outer layer.

43. The tablet of claim 42, wherein above 50% of the total carbidopa present in the tablet is in the outer layer.

44. The tablet of claim 43, wherein above 70% of the total carbidopa present in the tablet is in the outer layer.

45. The tablet of any one of claims 3 to 5, wherein the tablet comprises about 342.0 mg total of levodopa ethyl ester or derivative or pharmaceutically acceptable salt thereof, and about 75.0 mg total of carbidopa.

46. The tablet of any one of claims 3 to 5, wherein the tablet comprises about 228.0 mg total of levodopa ethyl ester or derivative or pharmaceutically acceptable salt thereof, and about 50.0 mg total of carbidopa.

47. The tablet of any one of claims 3 to 5, wherein the tablet comprises about 114.0 mg total of levodopa ethyl ester or derivative or pharmaceutically acceptable salt thereof, and about 25.0 mg total of carbidopa.

48. The tablet of any one of claims 3 to 5, wherein the tablet comprises about 57.0 mg total of levodopa ethyl ester or derivative or pharmaceutically acceptable salt thereof, and about 12.5 mg total of carbidopa.

49. The tablet of claim 4, wherein in the inner core
the granulated admixture comprises sodium lauryl sulfate as the ionic surfactant and further comprises above 0 mg up to about 150 mg of a microcrystalline cellulose and from about 1 mg to about 50 mg of a hydroxypropylcellulose,
the 4 mg up to about 400 mg levodopa ethyl ester or derivative or pharmaceutically acceptable salt thereof is present, and
the inner core excipient component comprises from about 2.5 mg up to about 245 mg hydroxypropylmethylcellulose, above 0 up to about 150 mg of a microcrystalline cellulose, from about 1 mg to about 10 mg of a micron-sized silica and from about 1 mg to about 30 mg sodium stearyl fumarate and/or magnesium stearate,
and wherein in the outer layer
the granulated carbidopa is present in an amount from about 1 mg up to about 50 mg, and is present in a granulated admixture with 0 mg up to about 300 mg of a microcrystalline cellulose, from above 0 mg up to about 150 mg of a partially pregelatinized maize starch and from above 0 mg up to about 50 mg of a hydroxypropylcellulose,
about 114 mg levodopa ethyl ester is present, and
the outer layer excipient component comprises about 80 mg of a microcrystalline cellulose, about 4 mg of a partially pregelatinized maize starch, about 3 mg of a micron-sized silica and from about 7.5 up to about 8.0 mg sodium stearyl fumarate.

50. The tablet of claim 49, wherein in the inner core
the granulated admixture comprises above 0 mg sodium lauryl sulfate, about 45 mg of a microcrystalline cellulose, about 7 mg of a hydroxypropylcellulose, and about 22 mg carbidopa;
about 114 mg levodopa ethyl ester is present; and
the inner core excipient component comprises about 35 mg of a hydroxypropylmethylcellulose, about 25 mg of a microcrystalline cellulose, about 3 mg of a micron-sized silica and about 5 mg of sodium stearyl fumarate,
and wherein in the outer layer
about 32 mg granulated carbidopa is present in a granulated admixture with about 24 mg of a microcrystalline cellulose, about 20 mg of a partially pregelatinized maize starch, and about 7 mg of a hydroxypropylcellulose.

51. A tablet which comprises
an inner core formulated for controlled release comprising a mixture of
(a) a granulated admixture of above 0 mg up to about 100 mg carbidopa, from about 0.03 mg to about 75 mg of a surfactant and at least one excipient,
(b) from about 4 mg up to about 400 mg levodopa ethyl ester or a derivative or a pharmaceutically acceptable salt thereof, and
(c) an inner core excipient component;
and an outer layer encapsulating the inner core and formulated for immediate release comprising a mixture of
(i) a granulated admixture of from about 1 mg up to about 75 mg carbidopa and at least one excipient; and
(ii) from about 5 mg up to about 300 mg levodopa ethyl ester or a derivative or a pharmaceutically acceptable salt thereof, and
(iii) an outer layer excipient component.

52. The tablet of claim 1, wherein the surfactant is an ionic or an aionic surfactant.

53. A process for manufacturing the tablet of claim 1, comprising
(A) preparing a granulated admixture of a decarboxylase inhibitor and a surfactant;
(B) mixing the granulated admixture from of step (A) with levodopa ethyl ester or a derivative or a pharmaceutically acceptable salt thereof;
(C) compressing the mixture from step (B) to form the inner core;
(D) separately mixing a granulated decarboxylase inhibitor with levodopa ethyl ester or a derivative or a pharmaceutically acceptable salt thereof;
(E) compressing the mixture of step (D) over the inner core formed in step (C) to form an outer layer encapsulating the inner core so as to thereby manufacture the tablet.

54. A process for manufacturing the tablet of claim 3, comprising
(A) preparing a granulated admixture of carbidopa, and a surfactant;
(B) mixing the granulated admixture from step (A) with an inner core excipient and levodopa ethyl ester or a derivative or a pharmaceutically acceptable salt thereof;
(C) compressing the mixture from step (B) to form the inner core;
(D) separately mixing carbidopa with an outer layer excipient compound and levodopa ethyl ester or a derivative or a pharmaceutically acceptable salt thereof;
(E) compressing the mixture of step (D) over the inner core formed in step (C) to form an outer layer encapsulating the inner core so as to thereby manufacture the tablet.

55. A process for manufacturing the tablet of claim 5, comprising
(A) preparing a granulated admixture of carbidopa and a surfactant;
(B) mixing the granulated admixture from of step (A) with an inner core excipient component and levodopa ethyl ester or a derivative or a pharmaceutically acceptable salt thereof;
(C) compressing the mixture from step (B) to form the inner core;
(D) separately mixing granulated carbidopa with an outer layer excipient and levodopa ethyl ester or a derivative or a pharmaceutically acceptable salt thereof;
(E) compressing the mixture of step (D) over the inner core formed in step (C) to form an outer layer encapsulating the inner core so as to thereby manufacture the tablet.

56. A process for manufacturing the tablet of claim 51, comprising
(A) preparing a granulated admixture of above 0 mg up to about 100 mg carbidopa and from about 0.03 mg to about 75 mg of an ionic surfactant and at least one excipient;
(B) mixing the granulated admixture from of step (A) with from about 4 mg up to about 400 mg levodopa ethyl ester or a derivative or a pharmaceutically acceptable salt thereof and an inner core excipient component;
(C) compressing the mixture from step (B) to form the inner core;
(D) separately granulating from about 1 mg to about 75 mg carbidopa with at least one excipient to form a granulated admixture;
(E) mixing the granulated admixture from step (D) with an outer layer excipient and from about 5 mg up to about 300 mg levodopa ethyl ester or a derivative or a pharmaceutically acceptable salt thereof;
(F) compressing the mixture of step (E) over the inner core formed in step (C) to form an outer layer encapsulating the inner core so as to thereby manufacture the tablet.

57. A method of treating a subject suffering from a disease selected from the group consisting of Parkinson's disease, senile dementia, dementia of the Alzheimer's type, a memory disorder, depression, hyperactive syndrome, an affective illness, a neurodegenerative disease, a neurotoxic injury, brain ischemia, a head trauma injury, a spinal trauma injury, schizophrenia, an attention deficit disorder, multiple sclerosis, withdrawal symptoms, epilepsy, convulsions and seizures, which comprises administering to the subject the tablet of claim 1, 3, or 51 in an amount effective to treat the disease.

58. The method of claim 57, wherein the disease is Parkinson's disease.
